(19) United States
(12) Patent Application Publication
(10) Pub. No.: US 2009/0024079 A1
(43) Pub. Date: Jan. 22, 2009

(54) METHOD FOR CONTROLLING INSULIN PUMP USING BLUETOOTH PROTOCOL

(76) Inventor: SOO Bong CHOI, CHUNGJU-SHI (KR)

Correspondence Address: HOLME ROBERTS & OWEN LLP, 1700 LINCOLN STREET, SUITE 4100, DENVER, CO, 80203, US

(21) Appl. No.: 12/182,812

(22) Filed: Jul. 30, 2008

Related U.S. Application Data
(63) Continuation of application No. 10/852,483, filed on May 24, 2004.

(51) Int. Cl. A61M 5/168

(52) U.S. Cl. 604/66; 455/41.3

(57) ABSTRACT

A method for controlling an insulin pump using the Bluetooth protocol. The method comprises allocating IDs to insulin pumps and blood sugar level measuring devices, respectively; checking, in a corresponding insulin pump, whether or not blood sugar level data is inputted from a blood sugar level measuring device having a corresponding ID; cumulating inputted data and judging whether or not abnormality occurs; generating a command to allow the insulin pump to operate as it is, when abnormality did not occur; generating a command to change an insulin injection amount of the insulin pump when abnormality occurred, and then, returning to the third step; and determining a system to be out of order when data is not inputted in the checking step, and generating an alarm so that a qualified person can visit a corresponding patient to have a trouble to be directly addressed.

### CROSS-REFERENCE TO RELATED APPLICATIONS

This application is a continuation of U.S. patent application Ser. No. 10/852,483 filed on May 24, 2004 and entitled “METHOD FOR CONTROLLING INSULIN PUMP USING BLUETOOTH PROTOCOL”. The entire disclosure of U.S. patent application Ser. No. 10/852,483 is considered to be part of the disclosure of the present application and is hereby incorporated by reference in its entirety.

### STATEMENT REGARDING FEDERALLY SPONSORED RESEARCH OR DEVELOPMENT

Not Applicable

### BACKGROUND OF THE INVENTION

The present invention relates to a method for controlling an insulin pump using the Bluetooth protocol, wherein Bluetooth chips capable of radio communication are respectively built in the insulin pump and a blood sugar level measuring device to drive the insulin pump through intercommunication and a separate Bluetooth communication device is linked with a server computer to control individual insulin pumps and blood sugar level measuring devices.

### DESCRIPTION OF THE RELATED ART

The diabetes is regarded, which is incidental to civilization. One billion or more persons among a worldwide population of about sixty billions are suffering from the diabetes, and it is estimated in Korea that approximately two millions of people and ten percents of medical patients are diabetics. So far, the diabetes is regarded as a disease which is not completely cured but administrated to get better in its condition. If the administration is unsuccessful, a patient may lose his or her life due to various diabetes complications. In Korea, as a death rate owing to diabetes is increased to 11.5 person s per a hundred thousand people (statistics on 1990), the diabetes becomes an object of fear.

The diabetes is diagnosed when a blood sugar level exceeds 140 mg/dl on an empty stomach or is no less than 200 mg/dl two hours after meal. The exact cause of these abnormal increases of a blood sugar level is not yet known in the art. So far, it is known that the diabetes may result in when abnormality occurs in insulin functioning to regulate metabolism of glucose. Abnormality of insulin means that the beta cells of the pancreas which secrete insulin do not sufficiently produce insulin, thereby causing an insulin-lacking state, or that, while the beta cells of the pancreas normally secrete insulin, functionality of the insulin is diminished for some reasons in such a way as not to properly regulate metabolism of glucose, thereby increasing a blood sugar level due to the so-called insulin resistance. Methods for treating the diabetes are largely classified into diet, exercise, medicinal therapy, insulin injection, and pancreatic grafting.

Insulin injection is a treatment method used to an insulin-dependent diabetic patient but takes effect also on a non-insulin-dependent diabetic patient. When carrying out the insulin injection method, while it is a norm that insulin is injected once or twice a day, an amount of insulin secreted in the human body is not constant, that is, secretion of insulin is increased three times a day before and after meal and decreased except those times. Therefore, in the insulin injection method in which an amount of insulin corresponding to an average insulin secretion amount of the human body can not but be injected once or twice a day, insulin becomes deficient after meal to induce a hyperglycemic state but excessive in the night to induce a hypoglycemic state. Consequently, since insulin supply is abnormal, a health condition of the human body cannot but be deteriorated. Accordingly, it is to be readily understood that the existing insulin injection method cannot supply changing amounts of insulin in conformity with changes in the insulin secretion as in a normal person and therefore cannot be of help to the prevention of diabetes complications. Accordingly, as improved diabetes treatment techniques, there are disclosed in the art a portable insulin pump in which an insulin injection amount is adjusted by a computer to conform to the insulin secretion of a normal person, and a method for grafting beta cells of the pancreas.

Generally, an automatic syringe device (also called as an insulin pump, insulin syringe device, automatic insulin syringe device, and so forth) used for prolonged injection of liquid has a configuration in which push means for pushing a syringe piston is coupled to a housing accommodating an injection syringe. This type of automatic syringe device is disclosed in Japanese Utility Model Laid-open Publication No. Sho 52-3292 and U.S. Pat. No. 4,417,889. However, since this type of automatic syringe device is complicated in use, inconvenience is caused when an old or feeble person manipulates the automatic syringe device.

In order to solve such a disadvantage, the present applicant disclosed in Korean Patent No. 307191 an insulin pump which is convenient to use and has a compact design. Referring to FIG. 1, in the insulin pump, when a syringe is refilled with injection liquid after use, a rotating shaft can be removed from a housing in a manner such that a precise filling height can be easily set while being viewed with the naked eye and then the rotating shaft and a push plate can be coupled in place to the housing. The insulin pump includes an injection needle unit which employs a feeding tube 1 connected to a connector 2. The injection needle unit is assembled to a housing 120 by means of a cover 110 which is sealably coupled to an upper end of the housing 120 at one side of the housing 120. Under the cover 110, a syringe 21, a piston 122, piston push means 150, power transmission means 130, and a rotating shaft 131 adapted to drive the piston push means 150 by power transmitted from the power transmission means 130 are arranged in the housing 120. A key input unit 123 is also installed on the housing 120 and electrically connected to a control circuit provided in the housing 120 to control the power transmission means 130. A display 124 such as an LCD is also installed on the housing 120 in order to display a controlled state of the syringe device. At the other side of the housing 120, a battery cover 125 is coupled to the upper end of the housing 120 to fixedly hold a battery in the housing 120. A reset button 121 functions to generate a reset signal for the control circuit. The reference numeral 140 represents a bottom cover.

FIG. 2 is a block diagram illustrating a control circuit of the insulin pump shown in FIG. 1. The control circuit includes the key input unit 123 for generating a key signal, a control unit 170 having a microcomputer function to recognize a key input generated from the key input unit 123, the display 124 for outputting data corresponding to the recognized key input and displaying the data, and a ROM 165 for storing diverse data and programs. The control circuit also includes a motor drive unit 167 for driving a motor 168 under the control of the control unit 170 while controlling a rotating speed of the motor 168, and a photocoupler 169 for sensing the rotating speed of the motor 168. Preferably, the control unit 170 includes a pair of controllers, that is, a first controller 171 and a second controller 172, which have the same function, in order to maintain a desired function even when one of the controllers 171 and 172 is out of order. The controllers 171 and 172 have terminals P1 to P5 and terminals P1′ and P2′, respectively. These terminals are ports connected to data and/or bus lines, respectively. The motor 168 may be a stepping motor or a servo motor.

FIG. 3 is a cross-sectional view illustrating a blood sugar level measuring device 200 according to the conventional art. The blood sugar level measuring device 200 includes a measuring lamp 211 for measuring a blood sugar level, a control unit 210 for controlling the measuring lamp 211, recognizing a blood sugar level inputted from the measuring lamp 211 and conducting appropriate signal conversions, a housing 223 having a lamp hole 221 through which the measuring lamp 211 is fitted and an insertion groove 222 into which a measuring probe 230 is inserted, and a fixing protrusion 224 which is spring-biased in the housing 223 to fixedly hold the measuring probe 230 inserted into the insertion groove 222. The measuring probe 230 has a fitting hole 231 into which the fixing protrusion 224 is fitted, a light passage hole 233 which is defined at a position corresponding to the measuring lamp 211 when the measuring probe 230 is inserted into the insertion groove 222, and a measuring plate 235 which closes one end of the light passage hole 233. The reference numeral 240 represents a base member to which the housing 223 is secured.

FIG. 4 is a block diagram illustrating a control circuit of the blood sugar level measuring device shown in FIG. 3. The control circuit has a control unit 210 which functions to receive a command from a microcomputer 250 and a measurement of blood sugar level from the measuring lamp 211. The control unit 210 includes a digital/analog converter 212 for converting an output from a terminal P7 of the microcomputer 250 into an analog signal, a lamp driver 213 for driving a light emitting lamp element 211-1 of the measuring lamp 211 based on a converted signal output from the digital/analog converter 212, with the measuring lamp 211 composed of the light emitting lamp element 211-1 and a light receiving lamp element 211-2 which receives light emitted from the light emitting lamp element 211-1 and reflected by the measuring plate 235, a lamp signal receiver 214 for receiving and amplifying the light received by the light receiving lamp element 211-2 of the measuring lamp 211, and an analog/digital converter 215 for converting an output from the lamp signal receiver 214 into a digital signal and transmitting the digital signal to the terminal P7 of the microcomputer 250.

FIG. 5 is a time chart illustrating a relationship between blood sugar level and insulin injection amount with the lapse of time.

Meanwhile, as a concept of local area radio communication which replaces local area wire transmission or infrared-ray communication, the Bluetooth protocol has been proposed in the art by the company named Ericsson. While the Bluetooth communication as local area radio communication which enables two-way transmission of voice and data is expected to be widely used in the future in the field of a communication terminal, in these days, a technology for applying the Bluetooth communication method to a radio telephone by solving the problem provoked by ringing of the radio telephone at a public place has not yet been disclosed in the art. In this regard, an attempt to solve the problem is disclosed in Korean Patent No. 341988 as illustrated in FIGS. 6 and 7.

FIG. 6 is a schematic diagram illustrating a Bluetooth communication device and a radio telephone which is capable of Bluetooth radio communication with the Bluetooth communication device. The Bluetooth communication device 300 installed at a public place functions to find all radio telephones 400 which exist within a distance enabling the Bluetooth radio communication and implement through radio communication a controlling operation for intended conversion from an alarm mode into a manner mode. At this time, the Bluetooth communication device 300 serves as a master, and all radio telephones 400 which are within the distance enabling the Bluetooth radio communication serve as: slaves. The radio telephones 400 which can be controlled by the Bluetooth communication device 300 must be respectively equipped with the Bluetooth modules 410 by themselves.

FIG. 7 is a block diagram illustrating a control circuit of the Bluetooth communication device 300 shown in FIG. 6. The Bluetooth communication device 300 includes an RF transmitter 310, an RF receiver 320, a baseband processor 330 and a communication controller 340. The RF transmitter 310, RF receiver 320 and baseband processor 330 constitute a transmitter/receiver unit 350.

The RF transmitter 310 modulates a data packet which is generated in the baseband processor 330 to be radio-transmitted, into a preset frequency band, and then amplifies and outputs the modulated data packet.

The RF receiver 320 maximally suppresses amplification of noise of a received frequency signal, amplifies a signal having the preset frequency band, modulates the signal to a low frequency band, and then outputs the signal having the low frequency band to the baseband processor 330.

The baseband processor 330 changes various HCI (host control interface) data packets outputted from the communication controller 340 into packet formats by adding access codes and headers to the data packets, changes again the packet formats into predetermined data packets for radio transmission, radio-transmits the predetermined data packets through the RF transmitter 310 at the preset frequency band, changes the data packets received from the RF receiver 320 into the HCI packets, and then outputs the changed HCI packets to the communication controller 340.

The communication controller 340 controls the entire operations of the Bluetooth communication device 300. When receipt of inquiry and answer messages (inquiry and answer data packets) from the radio telephones serving as the slaves, which are inputted from the baseband processors 330, is sensed, the communication controller 340 establishes connections with the respective radio telephones, and then controls the respective radio telephones to compulsorily convert the alarm mode into the manner mode.

The above-described technologies are independently used in their respective fields of use. In particular, since radio communication cannot be implemented between an insulin pump and a blood sugar measuring device, a patient should separately use the insulin pump and the blood sugar measuring device, so that inconvenience is caused to a patient who uses both of the insulin pump and the blood sugar measuring device.

### SUMMARY OF THE INVENTION

Accordingly, the present invention has been made in an effort to solve the problems occurring in the related art, and an object of the present invention is to provide a method for controlling an insulin pump using the Bluetooth protocol, which enables radio communication between the insulin pump and a blood sugar level measuring device, so that the insulin pump can be operated in real time in response to a measurement of the blood sugar level measuring device.

Another object of the present invention is to provide a method for controlling an insulin pump using the Bluetooth protocol, wherein Bluetooth chips are respectively built in the insulin pump and a blood sugar level measuring device to allow intercommunication between the insulin pump and the blood sugar level measuring device, and a separate Bluetooth communication device is provided to communicate with the insulin pump and the blood sugar level measuring device, so that a doctor or a nurse can control the Bluetooth communication device to conduct remote manipulation over the insulin pump and the blood sugar level measuring device.

In order to achieve the above objects, in the present invention, Bluetooth chips are respectively built in an insulin pump and a blood sugar level measuring device so that the insulin pump and the blood sugar level measuring device can communicate with each other through the Bluetooth chips.

Further, in the present invention, Bluetooth chips are respectively built in insulin pumps and blood sugar level measuring devices, and a separate Bluetooth communication device is provided to communicate with the plurality of insulin pumps and blood sugar level measuring devices, so that a doctor or a nurse can control the Bluetooth communication device through a computer to allow even an overseas patient to be appropriately placed under medical care.

According to one aspect of the present invention, there is provided a method for controlling an insulin pump using the Bluetooth protocol, comprising the steps of: preparing insulin pumps and blood sugar level measuring devices in which Bluetooth chips are built in, respectively, a Bluetooth communication device which is separate from the Bluetooth chips and has a transmitting/receiving unit and a communication control unit, and a computer which comprises a CPU having built therein a Bluetooth chip adapted to transmit and receive a signal to and from the Bluetooth communication device, a monitor and a keyboard; allocating IDs to insulin pumps and blood sugar level measuring devices, respectively; checking, in a corresponding insulin pump, whether or not blood sugar level data is inputted from a blood sugar level measuring device having a corresponding ID; cumulating inputted data and judging whether or not abnormality occurs; generating a command to allow the insulin pump to operate as it is, when abnormality did not occur; generating a command to change an insulin injection amount of the insulin pump when abnormality occurred, and then, returning to the third step; and determining a system to be out of order when data is not inputted in the checking step, and generating an alarm so that a qualified person can visit a corresponding patient to have a trouble to be directly addressed.

According to another aspect of the present invention, there is provided a method for controlling an insulin pump using the Bluetooth protocol, comprising the steps of: preparing insulin pumps and corresponding blood sugar level measuring devices in which Bluetooth chips are built in, respectively, to allow intercommunication between the insulin pumps and the corresponding blood sugar level measuring devices, a Bluetooth communication device which has a central controlling function, and a computer which comprises a CPU having built therein a Bluetooth chip and communicates with the Bluetooth communication device to transmit and receive data; generating a command to initially conduct a basic set mode; checking whether or not blood sugar level data is inputted from a corresponding blood sugar level measuring device; generating a command to drive a corresponding insulin pump to thereby inject an amount of insulin which is appropriate to the inputted blood sugar level data when the blood sugar level data is inputted from the corresponding blood sugar level measuring device; judging whether or not a doctor mode is required; generating a command to first drive the corresponding insulin pump in the doctor mode when the doctor mode is required; and returning to the command generating step to initially conduct the basic set mode, when the doctor mode is completed.

### BRIEF DESCRIPTION OF THE DRAWINGS

The above objects, and other features and advantages of the present invention will become more apparent after a reading of the following detailed description when taken in conjunction with the drawings, in which:

FIG. 1 is a perspective view illustrating an insulin pump to which the present invention is applied;

FIG. 2 is a block diagram illustrating a control circuit of the insulin pump shown in FIG. 1;

FIG. 3 is a cross-sectional view illustrating a blood sugar level measuring device according to the conventional art;

FIG. 4 is a block diagram illustrating a control circuit of the blood sugar level measuring device shown in FIG. 3;

FIG. 5 is a time chart illustrating a relationship between blood sugar level and insulin injection amount with the lapse of time;

FIG. 6 is a schematic diagram illustrating a typical example of communication using the Bluetooth protocol;

FIG. 7 is a block diagram illustrating a control circuit of a Bluetooth communication device shown in FIG. 6;

FIG. 8 is a block diagram for explaining a method for controlling an insulin pump using the Bluetooth protocol in accordance with an embodiment of the present invention;

FIG. 9 is a block diagram illustrating a control circuit of an insulin pump according to the present invention;

FIG. 10 is a block diagram illustrating a control circuit of a blood sugar level measuring device according to the present invention;

FIG. 11 is a block diagram illustrating a state in which insulin pumps and blood sugar level measuring devices communicate with a server computer via a Bluetooth communication device;

FIG. 12 is a flowchart of a program implemented in the control circuit of the insulin pump according to the present invention; and

FIG. 13 is a flowchart of a program implemented in the server computer according to the present invention.

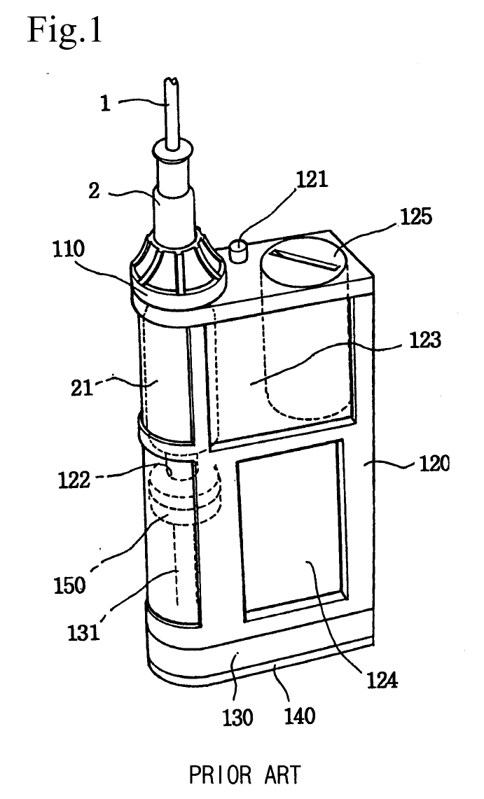

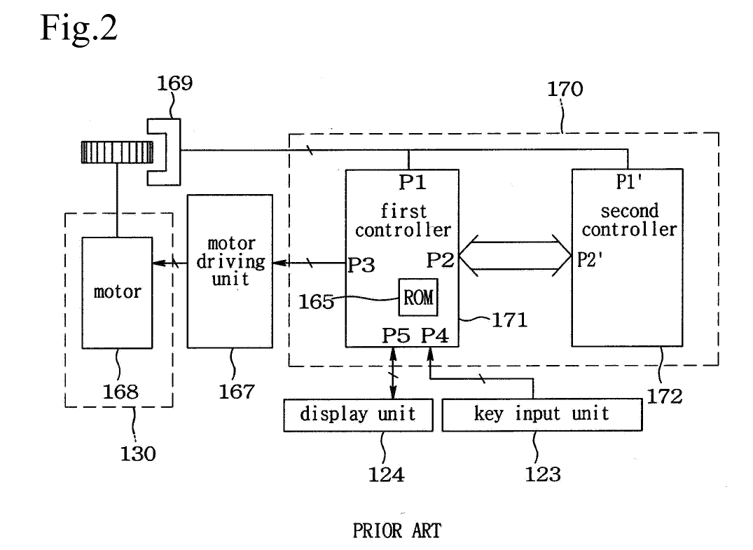

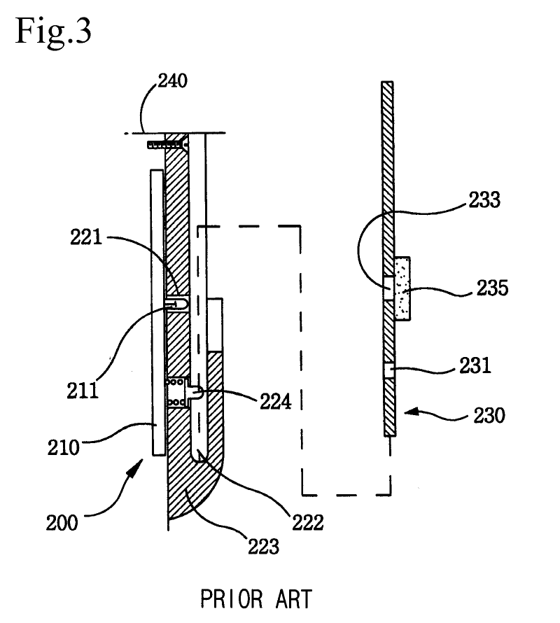

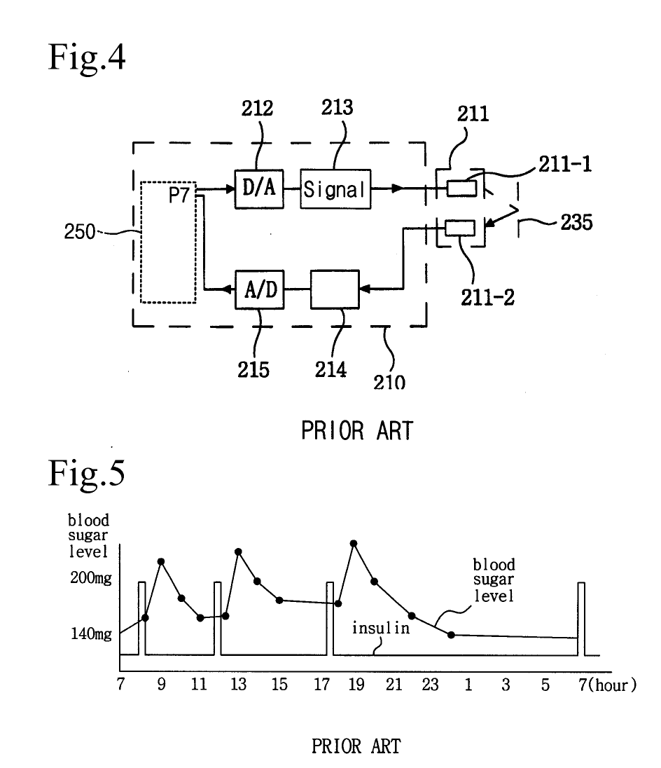

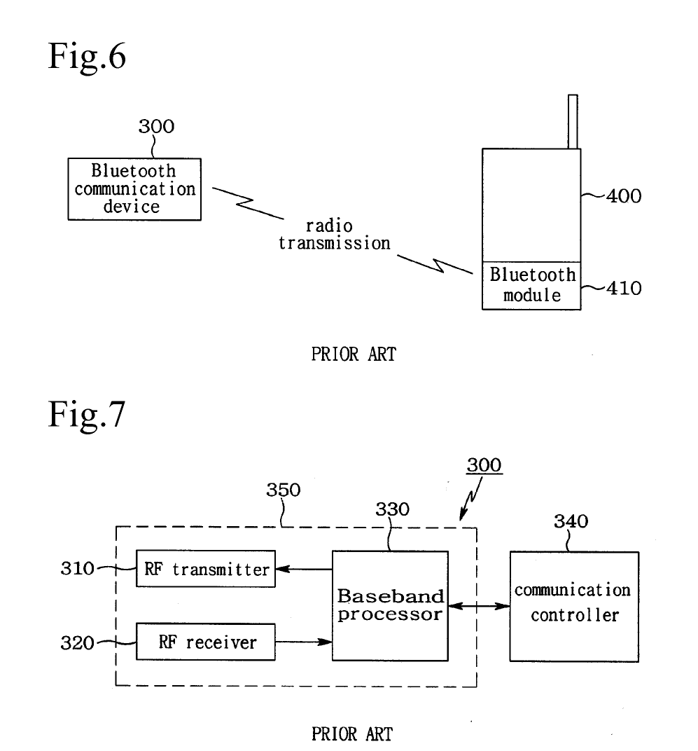

### DETAILED DESCRIPTION OF PREFERRED EMBODIMENTS

Reference will now be made in greater detail to a preferred embodiment of the invention, an example of which is illustrated in the accompanying drawings. Wherever possible, the same reference numerals will be used throughout the drawings and the description to refer to the same or like parts.

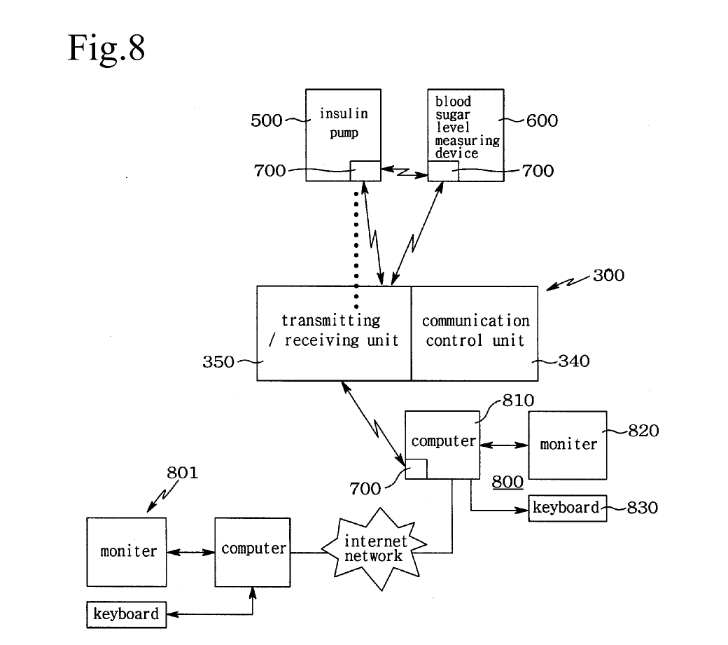

FIG. 8 is a block diagram for explaining a method for controlling an insulin pump using the Bluetooth protocol in accordance with an embodiment of the present invention. According to the present invention, Bluetooth chips 700 are built in an insulin pump 500 and a blood sugar level measuring device 600, respectively. Separately from the Bluetooth chips 700, a Bluetooth communication device 300 is provided. The Bluetooth communication device 300 has a transmitting/receiving unit 350 and a communication control unit 340. A computer 800 comprises a CPU 810 having built therein a Bluetooth chip 700 adapted to transmit and receive a signal to and from the Bluetooth communication device 300, a monitor 820 and a keyboard 830.

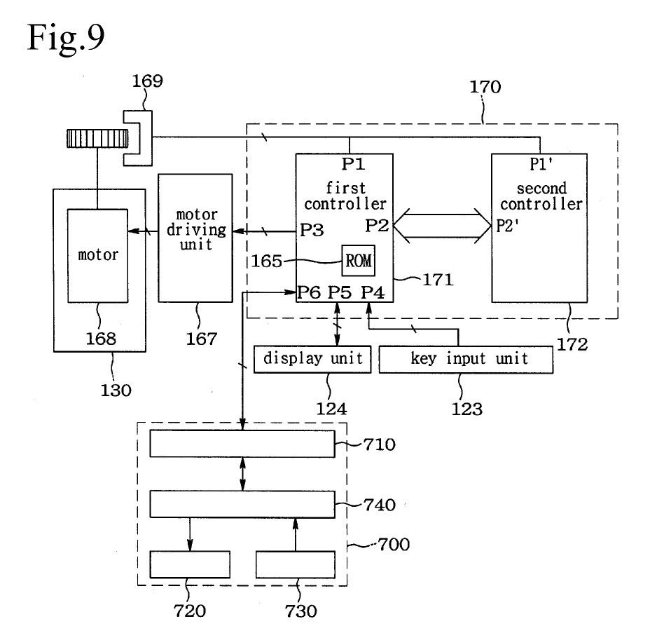

FIG. 9 is a block diagram illustrating a control circuit of an insulin pump according to the present invention. The control circuit includes a key input unit 123 for generating a key signal to drive the insulin pump, a control unit 170 having a microcomputer function to recognize a key input generated from the key input unit 123, a display 124 for outputting data corresponding to the recognized key input and displaying the data, and a ROM 165 for storing diverse data and programs. The control circuit further includes a motor drive unit 167 for driving a motor 168 under the control of the control unit 170 while controlling a rotating speed of the motor 168 for driving a syringe piston, and a photocoupler 169 for sensing the rotating speed of the motor 168. According to the present invention, a function for recognizing and controlling the Bluetooth chips is added as a program to the control unit 170 for controlling the insulin pump. The Bluetooth chip 700 is connected to a terminal of the control unit 170 to be controlled thereby.

The Bluetooth chip 700 comprises a microcomputer 710, an RF transmitter 720, an RF receiver 730 and a baseband processor 740. The microcomputer 710 receives a command from the control unit 170 and transmits data to the control unit 170. The RF transmitter 720 Modulates, in response to a command from the microcomputer 710, a signal which is generated by adding a header, etc. to a data packet and is transmitted from the baseband processor 740, and then, outputs an RF signal. The RF receiver 730 detects and receives a signal transmitted from another Bluetooth chip 700. The baseband processor 740 changes a command (data packet) from the microcomputer 710 into a transmission data packet to be radio-transmitted, by adding a header, etc. to the command, and then, outputs the transmission data packet to the RF transmitter 720. Further, the baseband processor 740 recognizes, from a received signal, an ID and data of a transmitter, changes the received signal into a data packet, and then, outputs the changed data packet to the microcomputer 710.

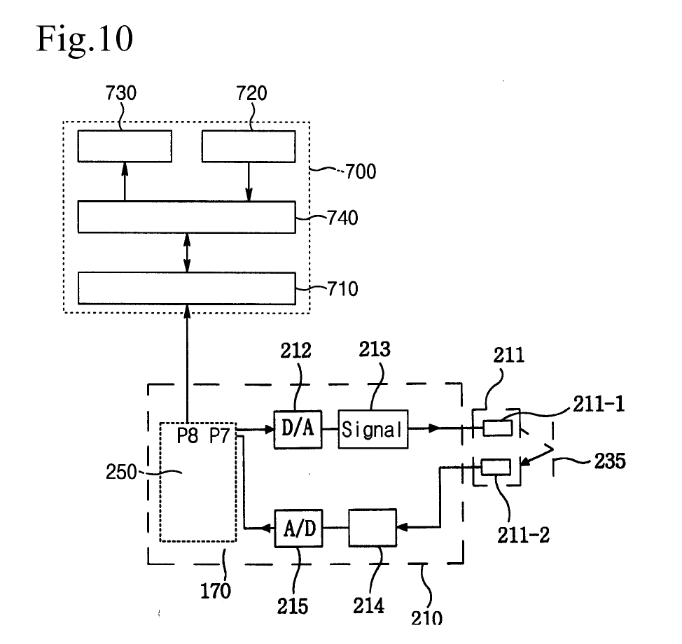

FIG. 10 is a block diagram illustrating a control circuit of a blood sugar level measuring device according to the present invention. The control circuit includes a microcomputer 250, a digital/analog converter 212 for driving a measuring lamp 211 in response to a command from the microcomputer 250, a lamp driver 213, a lamp signal receiver 214 for recognizing a measurement from the measuring lamp 211, and an analog/digital converter 215 for converting a signal outputted from the lamp signal receiver 214 into a digital signal and transmitting the digital signal to the microcomputer 250.

The Bluetooth chip 700 is connected to a terminal P8 of the microcomputer 250. A function for transmitting and receiving data to and from the Bluetooth chip 700 is added as a program to the microcomputer 250. The microcomputer 250 receives a signal from input means for driving the insulin pump. Since the Bluetooth chip 700 has the same configuration as that shown in FIG. 9, further detailed description thereof will be omitted herein.

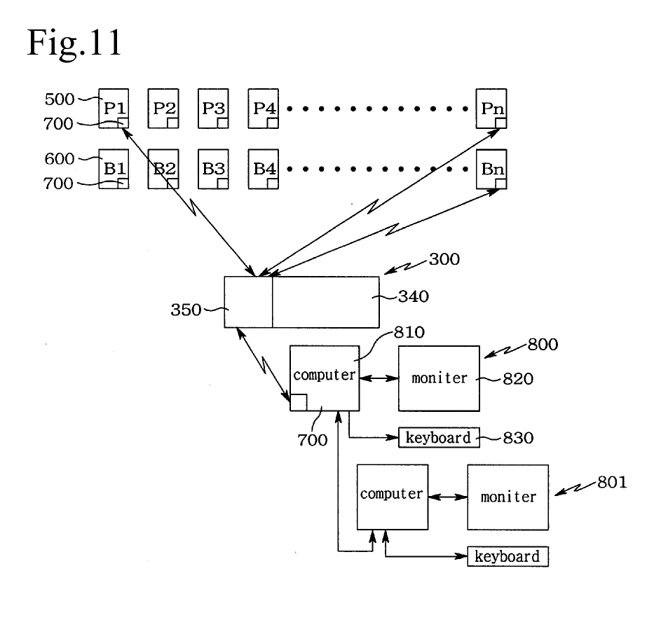

FIG. 11 is a block diagram illustrating a state in which insulin pumps and blood sugar level measuring devices communicate with a server computer via a Bluetooth communication device. Bluetooth chips 700 are respectively built in a plurality of insulin pumps P1-Pn and a plurality of corresponding blood sugar level measuring devices B1-Bn to allow intercommunication therebetween. Also, a Bluetooth communication device 300 having a central controlling function is provided. The Bluetooth communication device 300 has a transmitting/receiving unit 350 and a communication control unit 340. The transmitting/receiving unit 350 communicates with a computer 800 having a CPU 810 in which a Bluetooth chip 700 is built, to transmit and receive data. The reference numeral 820 designates a monitor, 830 a keyboard and 801 a personal computer.

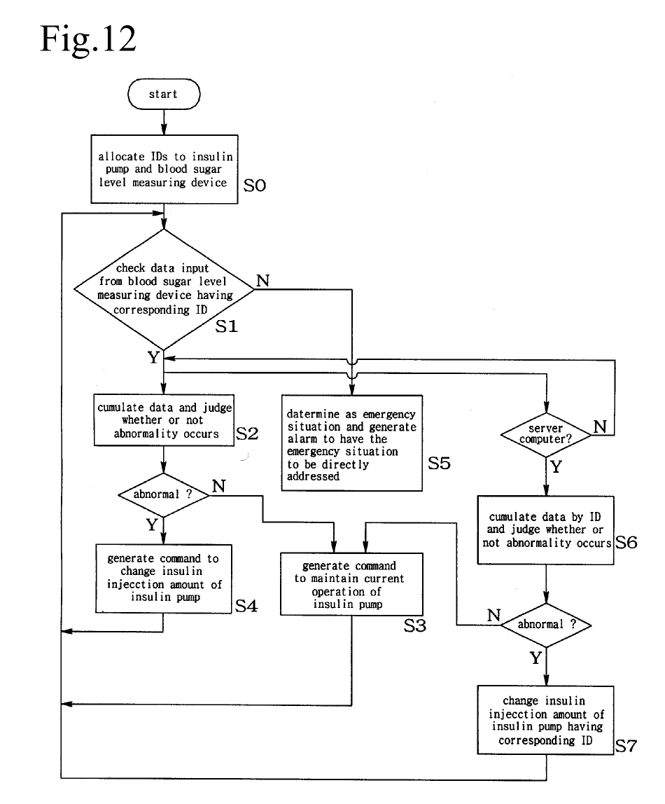

FIG. 12 is a flowchart of a program implemented in the control unit 170 of the insulin pump 500 in a manner such that data from the insulin pump 500 and the blood sugar level measuring device 600 is recognized to operate the insulin pump 500. First, as a preceding step, IDs are allocated to insulin pumps 500 and blood sugar level measuring devices 600, respectively (S0). Then, it is checked whether or not blood sugar level data is inputted from the blood sugar level measuring device having a corresponding ID (S1). Inputted data is cumulated and it is judged whether or not abnormality occurs (S2). If abnormality did not occur, a command is generated to allow the insulin pump to operate as it is (S3). If abnormality occurred, a command is generated to change an insulin injection amount of the insulin pump and then, the program is returned to step S1 (S4). If data is not inputted in step S1, the system is determined to be out of order, and an alarm is generated so that a qualified person can visit a corresponding patient to have a trouble to be directly addressed (S5).

In the case that the insulin pump to which the blood sugar level data is inputted in step S1 is established as a server, after step S1, blood sugar level data measured in the respective blood sugar level measuring devices are separately cumulated, and it is judged whether or not abnormality occurs (S6). If it is judged in step S6 that abnormality occurred, a command is generated to change an insulin injection amount of a corresponding insulin pump (S7). If abnormality did not occur, the program is returned to step S3.

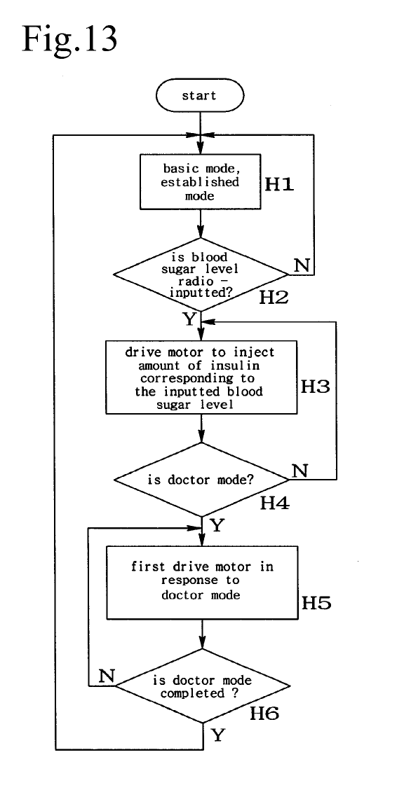

FIG. 13 is a flowchart of a program implemented in the CPU 810 of the server computer 800 according to the present invention, which receives data from the Bluetooth communication device 300 and generates an appropriate command. Initially, a command is generated to conduct a basic setting mode (H1). It is checked whether or not blood sugar level data is inputted from a corresponding blood sugar level measuring device (H2). A command is generated to drive a corresponding insulin pump to thereby inject an amount of insulin which is appropriate to the inputted blood sugar level data when the blood sugar level data is inputted from the corresponding blood sugar level measuring device (H3). It is judged whether or not a doctor mode is required (H4). A command is generated to first drive the corresponding insulin pump in the doctor mode when the doctor mode is required (H5). The program is returned to step H1 when the doctor mode is completed (H6).

In the present invention constructed as mentioned above, conventional Bluetooth chips 700 are respectively built in the insulin pump 500 and the blood sugar level measuring device 600. Also, the Bluetooth communication device 300 which communicates with the insulin pump 500 and the blood sugar level measuring device 600, and the computer 800 which communicates with the Bluetooth communication device 300 and has a server function are provided.

In the present invention, due to the fact that the insulin pump 500 and the blood sugar level measuring device 600 which belong to the same user can be communicated with each other using the Bluetooth chips 700, when the user measures a blood sugar level using the blood sugar level measuring device 600, it is not necessary for the user to directly manipulate the insulin pump 500, and instead, an insulin injection amount is automatically regulated by the insulin pump 500 in conformity with a measurement of the blood sugar level, whereby user convenience is rendered. To this end, as shown in FIGS. 8 and 12, in a state wherein IDs are allocated to insulin pumps 500 and blood sugar level measuring devices 600, respectively, to allow intercommunication therebetween (S0), a blood sugar level measurement (of course, the user must measure a blood sugar level using the blood sugar level measuring device 600) from the blood sugar level measuring device 600 having the corresponding ID is received through the Bluetooth chip 700 (S1).

The insulin pump 500 accumulates the inputted blood sugar level data, compares the inputted blood sugar level with a reference value preset as a standard, based on temporariness or continuousness of the inputted data, and judges whether or not abnormality occurs (S2). If abnormality occurred, a command is generated to change an insulin injection amount of the corresponding insulin pump and inject the changed amount of insulin (S4). If data is not inputted in step S11, the system is determined to be out of order, and an alarm is generated (S5).

Meanwhile, in the present invention, a doctor can carry around the insulin pump which serves as a portable server. In this regard, in the case that the corresponding insulin pump is established as a server, after step S1, blood sugar level data measured in the respective blood sugar level measuring devices are separately cumulated by ID, and it is judged whether or not abnormality occurs in respective IDs (S6).

Successively, if it is judged in step S6 that abnormality occurred, a command is generated and transmitted through the Bluetooth communication device 300 to drive the corresponding insulin pump 500 to thereby change an insulin injection amount of the corresponding insulin pump which is already set for the corresponding ID (S7).

In the meanwhile, in the present invention, if the server computer 800 is connected with the personal computer 801 through the Internet network as shown in FIG. 8, even an overseas doctor can easily regulate an insulin injection amount for a domestic patient. Of course, to this end, their IP addresses must be shared with each other, or a program for linking the computer 800 with the Bluetooth communication device must be installed to allow the computer 800 to operate as a server.

Moreover, in the present invention, as can be readily seen from FIGS. 11 and 13, IDs can be respectively allocated to a insulin pump 500; P1 and a corresponding blood sugar level measuring device 600; B1 so that they can be used in a pair. In this way, IDs can be allocated to the plurality of insulin pumps P1-Pn and the plurality of corresponding blood sugar level measuring devices B1-Bn to define a plurality of pairs, and the separate Bluetooth communication device 300 can be provided to enable intercommunication between the insulin pumps and the blood sugar level measuring devices which constitute the respective pairs. Since the intercommunication between the insulin pump 500 and the blood sugar level measuring device 600 is described with reference to FIGS. 8 and 12, detailed description thereof will be omitted herein. While the insulin pump 500 and the blood sugar level measuring device 600 are communicated with each other through the Bluetooth communication device 300, the Bluetooth communication device 300 enables the computer 800 having the CPU 810 in which the Bluetooth chip 700 is built, to serve as a server.

Initially, a command is generated by the CPU 810 of the computer 800 to drive the insulin pumps 500 through the Bluetooth communication device 300 and the Bluetooth chips 700 of the insulin pumps 500, so that the insulin pumps 500 can operate at the basic setting mode and their respective mode corresponding to the basic setting mode (H1). In this state, the computer 800 checks whether or not blood sugar level data is inputted from a corresponding blood sugar level measuring device 600, for example B 1, through the Bluetooth communication device 300 (H2). Then, the computer 800 compares the inputted blood sugar level with the stored reference value using the installed program, and generates a command through the Bluetooth communication device 300 to drive a corresponding insulin pump 500 to thereby inject an amount of insulin which is appropriate to the inputted blood sugar level data when the blood sugar level data is inputted from the corresponding blood sugar level measuring device (H3).

It is judged whether or not a doctor mode is required in step H3 (H4). If the doctor mode is required, a key input generated, as a prescription, through the keyboard 830 by the doctor is received, and the corresponding insulin pump is first operated to inject a corresponding amount of insulin (H5).

In the doctor mode, the doctor may set a time. In this regard, if a measurement is normal, a command is generated to conduct again the basic setting mode. Accordingly, if the doctor mode is completed as desired, the program is returned to step H1 (H6).

As apparent from the above description, in the method according to the present invention, Bluetooth chips are respectively built in an insulin pump and a blood sugar level measuring device, so that the insulin pump can be operated in real time through intercommunication between the insulin pump and the blood sugar level measuring device.

Also, in the method according to the present invention, IDs are allocated to a plurality of insulin pumps and blood sugar level measuring devices, and a Bluetooth communication device is centrally installed to control intercommunication between the insulin pumps and the blood sugar level measuring devices. The centrally installed Bluetooth communication device is linked with a computer having a server function, to radio-receive respective measurements of blood sugar level and generate corresponding prescription commands for corresponding insulin pumps, so that insulin injection amounts for a plurality of patients can be centrally regulated without causing nurses to visit the patients, thereby improving patient treating efficiency and enabling prescription to be rapidly made.

Further, by connecting the server computer with the Internet network, even an overseas doctor can easily make a prescription for a domestic patient.

In the drawings and specification, there have been disclosed typical preferred embodiments of the invention and, although specific terms are employed, they are used in a generic and descriptive sense only and not for purposes of limitation, the scope of the invention being set forth in the following claims.

1-3. (canceled)

4. A system for controlling an insulin pump comprising:
a first Bluetooth chip built in the insulin pump;
a blood sugar level measuring device having a second Bluetooth chip that is communicated with the first Bluetooth chip in radio frequency (RF) communication;
a Bluetooth communication device having a transmitting/receiving unit and a communication control unit; and
a computer having a third Bluetooth chip that communicates with the Bluetooth communication device.

5. The system according to claim 1, wherein the insulin pump further comprises:
a control unit connected to the first Bluetooth chip, for controlling the first Bluetooth chip;
a key input unit for generating a key signal to drive the insulin pump, which is input to the control unit;
a display for outputting data corresponding to the recognized key input and displaying the data; and
a memory for storing various data and programs that includes a program for recognizing and controlling the first Bluetooth chip.

6. The system according to claim 4, wherein the first Bluetooth chip comprises:
a microcomputer for receiving a command from the control unit and transmits data to the control unit;
an RF transmitter for modulating, in response to the command from the microcomputer, a signal which is generated by adding a header to a data packet;
an RF receiver for receiving signals transmitted from the other Bluetooth chips; and
a baseband processor for converting a command from the microcomputer into a transmission data packet to be radio-transmitted, by adding a header to the command, and outputting the transmission data packet to the RF transmitter,
wherein the baseband processor recognizes, from a received signal, an ID and data of a transmitter, converts the received signal into a data packet, and then, outputs the converted data packet to the microcomputer.

7. The system according to claim 6, wherein the blood sugar level measuring device comprises:
a microcomputer connected to the second Bluetooth chip, for receiving a signal from the other Bluetooth chips;
a digital/analog converter for driving a measuring lamp in response to a command from the microcomputer;
a lamp driver;
a lamp signal receiver for recognizing a measurement from the measuring lamp; and
an analog/digital converter for converting a signal outputted from the lamp signal receiver into a digital signal and transmitting the digital signal to the microcomputer.

8. The system according to claim 7, wherein the second Bluetooth chip comprises:
a microcomputer for receiving a command from the control unit 170 and transmits data to the control unit;
an RF transmitter for modulating, in response to the command from the microcomputer, a signal which is generated by adding a header to a data packet;
an RF receiver for receiving signals transmitted from the other Bluetooth chips; and
a baseband processor for converting a command from the microcomputer into a transmission data packet to be radio-transmitted, by adding a header to the command, and outputting the transmission data packet to the RF transmitter,
wherein the baseband processor recognizes, from a received signal, an ID and data of a transmitter, converts the received signal into a data packet, and then, outputs the converted data packet to the microcomputer.

9. The system according to claim 4, further comprising a server connected via the Internet,
wherein the server is connected to other computers via the Internet, which can access the computer and control the insulin pump.

10. A system for controlling a plurality of insulin pumps comprising:
first Bluetooth chips which are built in the insulin pumps, respectively;
a plurality of blood sugar level measuring devices having second Bluetooth chips, respectively, which correspond to the plurality of insulin pumps and are communicated with the first Bluetooth chips in radio frequency (RF) communication;
a Bluetooth communication device having a transmitting/receiving unit and a communication control unit; and
a computer having a third Bluetooth chip that communicates with the Bluetooth communication device.

11. The system according to claim 10, wherein each of the insulin pumps further comprises:
a control unit connected to the first Bluetooth chip, for controlling the first Bluetooth chip;
a key input unit for generating a key signal to drive the insulin pump, which is input to the control unit;
a display for outputting data corresponding to the recognized key input and displaying the data; and
a memory for storing various data and programs including a program for recognizing and controlling the first Bluetooth chip.

12. The system according to claim 10, wherein each of the first Bluetooth chips comprises:
a microcomputer for receiving a command from the control unit and transmits data to the control unit;
an RF transmitter for modulating, in response to the command from the microcomputer, a signal which is generated by adding a header to a data packet;
an RF receiver for receiving signals transmitted from the other Bluetooth chips; and
a baseband processor for converting a command from the microcomputer into a transmission data packet to be radio-transmitted, by adding a header to the command, and outputting the transmission data packet to the RF transmitter,
wherein the baseband processor recognizes, from a received signal, an ID and data of a transmitter, converts the received signal into a data packet, and then, outputs the converted data packet to the microcomputer.

13. The system according to claim 12, wherein the blood sugar level measuring device comprises:
a microcomputer connected to the second Bluetooth chip, for receiving a signal from the other Bluetooth chips;
a digital/analog converter for driving a measuring lamp 211 in response to a command from the microcomputer;
a lamp driver;
a lamp signal receiver for recognizing a measurement from the measuring lamp; and
an analog/digital converter for converting a signal outputted from the lamp signal receiver into a digital signal and transmitting the digital signal to the microcomputer.

14. The system according to claim 13, wherein the second Bluetooth chip comprises:
a microcomputer for receiving a command from the control unit and transmits data to the control unit;
an RF transmitter for modulating, in response to the command from the microcomputer, a signal which is generated by adding a header to a data packet;
an RF receiver for receiving signals transmitted from the other Bluetooth chips; and
a baseband processor for converting a command from the microcomputer into a transmission data packet to be radio-transmitted, by adding a header to the command, and outputting the transmission data packet to the RF transmitter,
wherein the baseband processor recognizes, from a received signal, an ID and data of a transmitter, converts the received signal into a data packet, and then, outputs the converted data packet to the microcomputer.

15. The system according to claim 10, further comprising a server connected via the Internet,
wherein the server is connected to other computers via the Internet, which can access the computer and control the insulin pump.

16. A method for controlling an insulin pump having a Bluetooth chip connected to a blood sugar level device having a Bluetooth chip and a computer having a Bluetooth chip via RF communication, the method comprising:
allocating IDs to the insulin pump and blood sugar level measuring device having a Bluetooth chip, respectively;
checking whether blood sugar level data is input from a blood sugar level measuring device corresponding to an ID;
accumulating, when the blood sugar level data is input, the blood sugar level data and judging whether the blood sugar level data is in a normal state;
generating a command to allow the insulin pump to operate according to a predetermined mode when the blood sugar level data is in a normal state; and
generating a command to change an insulin injection amount of the insulin pump when the data is not in a normal state.

17. The method according to claim 16, further comprising:
ascertaining that the system for controlling an insulin pump is malfunctioned, when the blood sugar level data is not input; and
generating an alarm to notify an operator.

18. The method according to claim 17, when the blood sugar level data is input in a server, the method further comprising:
accumulating blood sugar level data measured in the respective blood sugar level measuring devices, and judging whether the respective blood sugar level data is in a normal state; and
generating a command to change an insulin injection amount of a corresponding insulin pump whose blood sugar level data is not a normal state.

19. The method according to claim 18, further comprising:
generating a command to allow the insulin pump to operate according to a predetermined mode when the blood sugar level data is in a normal state.

20. A method for controlling a plurality of insulin pumps having Bluetooth chips connected to a plurality of blood sugar level devices having Bluetooth chips and a computer having Bluetooth chips via RF communication, the method comprising:
setting the insulin pumps to operate in a basic mode at the beginning;
checking whether blood sugar level data is input from a corresponding blood sugar level measuring device;
generating, when the blood sugar level data is input, a command to drive an insulin pump corresponding to the corresponding blood sugar level measuring device to inject an amount of insulin to match the input blood sugar level data;
judging whether a doctor mode is required;
generating a commend to first drive the corresponding insulin pump in the doctor mode when the doctor mode is required; and
returning to the setting step when the doctor mode is completed.
